(19) United States
(12) Patent Application Publication
(10) Pub. No.: US 2023/0067150 A1
(43) Pub. Date: Mar. 2, 2023

(54) ANTIMICROBIAL COATING

(71) Applicant: THE REGENTS OF THE UNIVERSITY OF CALIFORNIA, Oakland, CA (US)

(72) Inventors: Oren Gotlib, Agoura Hills, CA (US); Karcher Morris, La Jolla, CA (US); Madhu Alagiri, San Diego, CA (US); Frank E. Talke, La Jolla, CA (US); Frederick E. Spada, Carlsbad, CA (US); Michael T. Tolley, La Jolla, CA (US); Adriane F. Minori, La Jolla, CA (US); Simonas Vaitkus, Morris Plains, NJ (US); Rafaela Simoes-Torigoe, La Jolla, CA (US)

(21) Appl. No.: 17/796,586

(22) PCT Filed: Jan. 29, 2021
(86) PCT No.: PCT/US2021/015919

§ 371 Date: Jul. 29, 2022

Related U.S. Application Data
(60) Provisional application No. 62/968,098, filed on Jan. 30, 2020.

(51) Int. Cl. A61L 29/16; A61L 29/10; A61L 29/06

(52) U.S. Cl. CPC A61L 29/16; A61L 29/106; A61L 29/06; A61L 2300/104; A61L 2300/102; A61L 2300/404

(57) ABSTRACT

An antimicrobial coating is formed from a biocompatible flexible polymer having incorporated therein an active material having a reducible form of silver, where at least a portion of the active material is exposed at the surface of the polymer. The coating can be applied to the surface of a catheter to inhibit bacterial growth and biofilm formation.

### RELATED APPLICATIONS

This application claims the benefit of the priority of U.S. Provisional Application No. 62/968,096, filed Jan. 30, 2020, which is incorporated herein by reference in its entirety.

### FIELD OF THE INVENTION

The present invention relates to a coating that induces an electrochemical/galvanic reaction on coated surfaces when exposed to an electrolyte composed of bodily fluids, resulting in an antimicrobial effect.

### BACKGROUND

Catheters are devices that controllably direct flow of bodily fluids from various passages inside the body to outside the body. A urinary catheter is intended to be deployed into the urinary tract of a patient with urinary incontinence. A Foley's urinary catheter has two separate lumens and ports: one set to flush urine from the bladder, and one set to inflate the balloon at the distal end of the catheter. The balloon is designed to inflate in the bladder and prevent the catheter from inadvertently slipping out from the patient. Aside from biocompatibility, the most important constraints are a smooth surface finish and proper flexibility. In other words, mechanical trauma and shear forces at the biomaterial-tissue interface should be kept to a minimum.

Implants are often associated with infection as they provide an excellent surface for bacterial colonization. During acute infection, bacteria rapidly proliferate and spread as unicellular organisms, however, in chronic infections they predominantly colonize body surfaces and tissues as multicellular aggregates, termed biofilms. In the latter case, planktonic, or free flowing/moving bacteria, adhere to a surface of interest and begin to colonize that surface. Control of infection typically begins with removal of the implant or foreign body followed by a cocktail of antibiotics and anticoagulants. In many cases it is an advantage for bacteria to adhere to surfaces. For example, attachment to horizontal surfaces stimulates growth of bacteria as organic material suspended in liquid settles. Another example would be that of a surface made of nutrient rich material. The disadvantages to adhering to surfaces, is immotility caused by the change in gene expression, which would inhibit the ability to search for nutrients and lead to death by starvation.

The Centers for Disease Control and Prevention, and the Department of Health and Human Resources report that urinary tract infections (UTIs) are the most common type of healthcare-associated infection reported to National Healthcare Safety Network. In fact, UTIs acquired in hospitals are 75% associated with urinary catheters. Between 15-25% of hospitalized patients receive urinary catheters during a regular hospital stay. It is estimated that hospitals spend over $1 billion (US) in managing catheter-associated urinary tract infections (“CAUTI”), which is primarily caused by prolonged catheterization. For a patient, the daily risk of developing a urinary tract infection increases by 3-7% each day that a catheter is present. For decades, the medical community has sought to develop improved antifouling and biocidal materials capable of being incorporated onto the surfaces of medical devices. The ability to maintain the sterility of a surface against the resistive nature of microorganisms would prevent thousands of patients from contracting life-threatening infections.

Bacteria have many strategies to invade the human body. Bacterial adhesion is highly dependent on the physical properties of the media, the surface, and the bacteria membrane. In bacteria/surface interactions, extracellular organelles operate in such a way as to create physical attachments of the cell body to a surface. Cells attach preferentially to hydrophilic materials when the surface energy of the bacterium is larger than the surface energy of the liquid in which they are suspended. Typically, the surface energy of bacteria is smaller than the surface energy of the liquid in which they suspend; a mismatch can allow cells to attach preferentially to hydrophobic materials.

Surface attachment facilitates antibiotic resistance by reducing the net negative surface charge of bacterial cells and enhancing the stability of the membrane. Most bacteria have a net negative surface charge and interact preferentially with positively charged surfaces. The effect can disappear in media with high concentrations of ions due to charge screening. This phenomenon is physiochemical, where water is lost at the interfacial layer as changes to the structure of surface molecules occur during the time the cell body repositions to maximize attachment to that surface.

To complicate matters, bacteria can also attach to surfaces that initially resist the attachment of cells. They do so by depositing a layer of proteins, environmental and innate, that condition the surface and nullify functional groups that would normally reduce adhesion. The dense packing of cells in bacterial “quorums” facilitates an increase in the concentration of secreted small-molecules, forming stronger gradients which transfer information between cells and trigger physiological changes. Quorum sensing and charge screening are abilities of bacteria that allow for modulation of the membrane charge, to better adapt to a surface during initial stages of biofilm formation.

Upon aggregating at a surface, adhered bacteria initiate expression of new phenotypes, the result of which changes them into biofilm. Biofilms are aggregates of bacteria that communicate and maintain proliferation via the secretion of an extracellular polymeric substance (EPS) in which they encompass themselves. This matrix-like network allows a bacterial colony to be able to respond to environmental stress that would normally inhibit proliferation. Stresses include but are not limited to external attack, physical conditions, and nutrient limitation. For example, EPS secretion provides protection from mechanical damage and shear stress caused by fluid flow. The EPS assists bacterial colonies in many ways that include improved adhesion, communication, and nutrient supply.

Numerous studies reveal that rather than focusing solely on planktonic bacteria, it is important to also focus on the prevention of the biofilm; slow growth of the biofilms can confer resistance. These studies have led to the definition of two main approaches to antimicrobial agents: antifouling and biocidal.

Antifouling materials are organized into two categories: hydrophilic materials and polyzwitterions. The former group inhibits fouling by forming a barrier of hydration on the surface that induces a type of steric repulsion. The latter group employs electrostatic and low surface energy. Both antifouling categories represent coatings that do not kill bacteria, but thermodynamically prevent attachment of bacteria and/or protein to the surfaces. On the other hand, biocidal materials are designed to kill microbes instead of minimizing their deposition. These materials are seen as more important because they protect the patients from infection and encrustation development. Clinically tested catheters use silver, a well-known biocide, or antibiotic coatings. While the coatings are most commonly used, other biocidal materials are currently being researched.

An alternative approach to surface coatings involves the use of electrical fields and currents. In 2009, Del Pozo et al. conducted a study with the aim to determine the effect of prolonged exposure to low intensity direct electrical current (DC) on Pseudomonas aeruginosa, S. aureus, and S. epidermidis biofilms. These infectious species are well known to be the causes of most UTIs. The investigators tested biofilm-covered TEFLON® coupons submerged in a slightly ionic media exposed to DC electrical current. The study's conclusion was that higher amperage currents correlated with increased inhibition of biofilm at every time point during the study. These results also demonstrated that electrical current can substantially reduce the viability of biofilm on a TEFLON® surface. The mechanism, referred to as the “electricidal effect”, resulted from one or a combination of disruption of the integrity of the bacterial membrane and generation of chlorine, oxygen, and/or hydrogen peroxide as a result of electrolysis.

Catheter-associated urinary tract infection is still prevalent in hospitals around the world. Bacteria are the ultimate survivors, able to groom surfaces to their liking and pass on genes that enable resistance to harsh environments. The need exists for materials that can be used to passively mitigate the transmission of infection. The design and analysis of materials that are able to synergistically work to inhibit the growth and proliferation of bacteria on medical devices is the key to infection control.

### BRIEF SUMMARY

According to embodiments of the invention, a surface film or coating applied to the surfaces of urinary catheters inhibits the growth and proliferation of biofilm on the surfaces of catheters and the urinary tract by inducing an electrochemical/galvanic reaction when exposed to an electrolyte composed of bodily fluids. These reactions produce small scale electric fields and potentials capable of generating an ambipolar diffusion of ions, resulting in passive micro-current and formation of hydrogen peroxide. Testing confirms that these reactions are harmful to microorganisms, particularly bacteria and the biofilms they can form. In some embodiments, the reaction reagents, i.e., active materials, are heavy metals, metal alloys, and/or metal oxides that occur naturally in the human body or are non-toxic to humans in low doses. Examples include magnesium, calcium, manganese, zinc, silver, and others. In in vitro studies, the films are effective in inhibiting growth of planktonic E. coli in synthetic urine. After exposure of the films to inoculated urine for six days, the biofilm was found to be reduced in concentration by five orders of magnitude relative to a control (p<0.05)—a clinically significant improvement.

The inventive composite films induce reactions between the solution and the active materials in the films. These reactions are similar to those that occur within a battery. Stored electrochemical potential is released during the reaction with the urine, producing low level electric fields and potentials capable of inducing an ambipolar diffusion of ions, resulting in passively generated micro-current and formation of hydrogen peroxide. The precise mechanism is not known. Without intending to be bound by a particular theory, it is believed that the film generates an entourage effect that involves different antimicrobial mechanisms at the interface between the solution and the surface, making it difficult for microorganisms to survive. These mechanisms may include one or more of micro-currents, electric fields, metal toxicity, changes in pH, and generation of hydrogen peroxide.

In an exemplary implementation, the interaction with zinc and silver in solution creates an interfacial environment that is potentially too chaotic for bacteria to thrive. At the surface of the urinary catheter, bacteria encounter changes in pH, micro-currents generated from the ions caught in electric fields, toxic metal compounds, and peroxides. The selected active materials are thermodynamically capable of coupling with an ensemble of atomic and molecular ions that exist in urine. These ions can react with active materials to produce other chemical species or potentially produce a passivation layer on the surface of the active materials that inactivates them. A key goal of the inventive approach is to effectively combine these active materials with commercial urinary catheter materials to produce a new material with surfaces capable of perpetuating the electrochemical reactions upon exposure to bodily fluids. Urologists have suggested that it would be a major accomplishment to design a catheter with surfaces that are able to maintain sterility for 30 days. This is in contrast to the observation that most catheter patients develop a bacterial infection over that period.

In one aspect of the invention, an antimicrobial coating is formed from a biocompatible flexible polymer having incorporated therein an active material having a reducible form of silver, wherein at least a portion of the active material is exposed at the surface of the polymer. In some embodiments, the flexible polymer is polydimethylsiloxane (PDMS). The active material may include silver oxide or silver chloride, and may further include one or more of zinc and silver. The active material may be incorporated into the polymer by forming a paste from a monomer, a cross-linker and a powder of the active material and thermally curing the paste. In some embodiments, the paste is filled into a mold configured to form a lumen.

In another aspect of the invention, a method for inhibiting growth and proliferation of biofilm on a medical device includes applying an antimicrobial coating formed from a biocompatible flexible polymer having incorporated therein an active material having a reducible form of silver to surfaces of the medical device, wherein the coating is configured to induce an electrochemical/galvanic reaction on the device surfaces when exposed to an electrolyte comprising bodily fluids.

In another aspect of the invention, a catheter lumen includes a molded biocompatible flexible polymer having incorporated therein an active material comprising a reducible form of silver, wherein at least a portion of the active material is exposed at the surface of the polymer, wherein the active material is configured to induce an electrochemical/galvanic reaction when exposed to an electrolyte comprising bodily fluids. In some embodiments, the flexible polymer is polydimethylsiloxane (PDMS). The active material may include silver oxide or silver chloride, and may further include one or more of zinc and silver. The active material may be incorporated into the polymer by forming a paste from a monomer, a cross-linker and a powder of the active material and thermally curing the paste. In some embodiments, the paste is filled into a mold configured to form the lumen.

### BRIEF DESCRIPTION OF THE DRAWINGS

FIG. 1 is a diagram illustrating the steps of an exemplary process sequence for implementing one embodiment of the invention by embedding metal electrodes in PDMS using a combination of soft and photo-lithography.

FIG. 2A diagrammatically illustrates a series of steps for using a mold to maintain AM at the surface of the catheter; FIG. 2B is a photograph showing an exemplary mold for forming two catheter sections.

FIG. 3 is a photograph of an exemplary (balloon-free) single lumen Ag/Ag₂O:PDMS catheter fabricated according to an embodiment of the invention.

FIG. 4 plots the concentration of hydrogen peroxide measured with respect to time for various active material composites and controls immersed in synthetic urine.

FIG. 5 provides photos of Zn/Ag₂O:PDMS composite samples before and after exposure to synthetic urine for twenty-four hours. The region on the right side of each image was unexposed while the region on the left side of each image shows white crystal residue observed after exposure.

FIG. 6 is a plot of potential difference between silver and zinc foil specimens in air-saturated aqueous media with respect to time.

FIG. 7 is a graph of contact angles measured for AM:PDMS composites.

FIG. 8 is a plot of absorbance OD600 with respect to time for selected samples. The more bacteria in solution the higher the absorbance.

FIG. 9 is a plot of biofilm concentration in CFU/mL growing on the surface of selected samples following 48 hours. Samples with higher concentrations perform worse in inhibiting biofilm.

FIG. 10 is a plot of absorbance OD600 with respect to time for selected samples over a 6-day period.

FIG. 11 is a plot of biofilm concentration in CFU/mL growing on the surface of selected samples following six days. Samples with higher concentrations perform worse in inhibiting biofilm.

FIG. 12 is a plot showing the zone of inhibited bacteria about the 8 mm disk of AM:PDMS films on LB agar plates coated with E. coli (n=3). Radius of zone is measured from center of disk.

### DETAILED DESCRIPTION OF EMBODIMENTS

The description of the materials and procedures used for preparation and testing, employs a number of acronyms and abbreviations that are generally well known among those in the art. To avoid ambiguity, Table 1 below provides a list of acronyms/symbols and their meanings as used herein.

TABLE 1

| Acronym | Meaning |
|---|---|
| AM | Active Material |
| AMR | Antimicrobial Resistance |
| BED | Bioelectric Dressing |
| CFU | colony forming unit |
| DI | deionized (water) |
| LB | Lysogeny Broth |
| PDMS | Polydimethylsiloxane |
| PETG | Polyethylene terephthalate glycol |
| PR | photoresist |

The embodiments disclosed herein relate to a surface film or coating applied to the surfaces of a urinary catheter for the purpose of inhibiting the growth and proliferation of biofilm on the surfaces of catheters and the urinary tract. The inventive coating induces an electrochemical/galvanic reaction when exposed to an electrolyte composed of bodily fluids. The unique interaction of zinc and silver in solution creates an interfacial environment that is potentially too chaotic for bacteria to manage. At the surface of the urinary catheter, bacteria are exposed to changes in pH, micro-currents generated from the ions caught in electric fields, toxic metal compounds, and peroxides. The selected active materials are thermodynamically capable of coupling with an ensemble of atomic and molecular ions that exist in urine. These ions can react with active materials, referred to herein as “AMs”, to produce other chemical species or potentially produce a passivation layer on the surface of the active materials that inactivates them. The inventive approach is directed to an effective combination of these active materials (AMs), with commercial urinary catheter materials to produce a new material with surfaces capable of perpetuating the electrochemical reactions when exposed to bodily fluids. Urologists have suggested that it would be a major accomplishment to design a catheter with surfaces that maintain sterility for up to 30 days based on the observation that nearly every catheter recipient tends to develop a bacterial infection by that time.

Catheters are typically formed from polymers based on their desirable physical and chemical properties as well as ease of manufacture. Unlike vulcanized natural rubber latex, polydimethylsiloxane (PDMS) is inert, non-cytotoxic, and flexible. PDMS is one of the most widely used silicon-based organic polymers due to its versatility, with mechanical properties that can be varied depending on how much cross-linker is used for the polymerization reaction. For this reason, PDMS was selected as the preferred substrate to combine with our active materials. As will be readily apparent to those in the art, other polymers known to be appropriate for medical applications, particularly for implants, may be substituted. Polymer additives, as are known in the art, may include fillers, reinforcements, release agents, internal lubricants, catalysts, impact and toughness modifiers, thermal and radiation stabilizers, plasticizers, pigments, coupling agents, and antistats. A commercial-off-the-shelf silver coated Foley catheter was used as a benchmark for the study.

Different methods to fabricate thin-film and flexible zinc-silver batteries were evaluated. Methods that can be used to form a composite of the active materials with PDMS include chemical adhesion, chemical-free adhesion, and in situ synthesis. Covalent bonding of silicone elastomers on metallic substrates is difficult due to an incompatible surface chemistry. Adhesion can be promoted by the use of chemical coupling agents such as: organotitanates, amide/imides, zircoaluminates, organosilanes, etc. Such approaches involve the use of toxic reagents and may not be optimal. Nandkumar et al. described use of a chemical solvent and dissolved silver to impregnate latex Foley catheters. The described method used only silver as an active ingredient, used a different flexible substrate (Latex), and still involved the use of strong reagents. Thus, the chemical-free adhesion and in situ synthesis method would generally be a more attractive method for making the zinc-silver PDMS composites. Anubha Goyal et al. prepared a homogenous mixture of metal salt, silicone elastomer and the curing agent to produce metal nanoparticle embedded PDMS films. During the curing process, the hardener simultaneously crosslinks the elastomer and reduces the metal salt to form nanoparticles in situ. Although the authors reported reduced bacteria concentrations in solution following exposure to the material, it appears that silver ions that were not reduced and have not agglomerated into nanoparticles are able to escape the surface of the polymer matrix and therefore are unable to sustain the desired electrochemical behavior. To expand on the phenomena, the bacteria are most likely being affected by residual silver ions, not by nanoparticle interaction. A suspension of particles, flakes or fibers in a thermoset polymer is considered to be a composite, especially when the mechanical properties are improved.

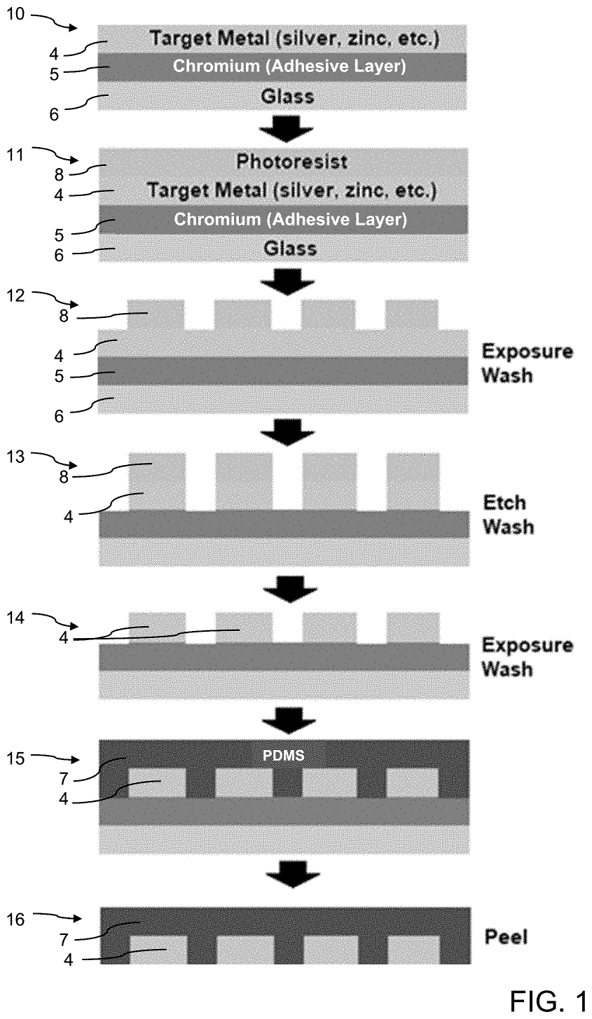

It is a common practice to fully embed fiber in resin when making a composite for mechanical loads. However, to achieve the results required of the inventive coating, the active material must be exposed at the surface of the polymer matrix rather than being fully embedded in it. This led to an approach that employed mechanical adhesion. In this case, a powder or metal thin film can be partially encased in PDMS and thermally bonded, leaving partial surfaces of the metal exposed. Authors Domin Koh et al. used a lithography-based method where a thin film of metal was evaporated onto chromium coated glass, etched into an electrode, and then coated in PDMS; the cured PDMS film could be separated from the chromium with the metal electrode imbedded at the surface. This process, described below with reference to FIG. 1, produces a detailed, controlled pattern of metal elements, e.g., silver and/or zinc, incorporated into the surface of PDMS.

Referring to FIG. 1, in step 10, the starting structure includes a target metal layer 4, which may be silver, zinc, or other appropriate metal adhered to a glass slide substrate 6 using an adhesive layer 5, which may be chromium. The metal layer may be formed by conventional techniques, e.g., evaporation or deposition, of thick film methods. In step 11, a layer of photoresist (PR) 8 is coated over the metal layer 4. In step 12, using a mask (not shown) the photoresist 8 is exposed to selectively protect the metal features to be defined. The unexposed PR is washed away and, in step 13, the metal 4 is selectively etched using an appropriate etchant (wet etch, plasma etch, or other, as is known in the art) to define the metal features 4. The PR 8 is rinsed in step 14, and, in step 15, the assembly is coated with a pre-polymer solution 7, in this example, PDMS, which is then exposed to an appropriate curing process. After curing, in step 16, the flexible polymer film 7 with embedded metal features 4 is peeled off of the glass substrate. The resulting film can then be rolled and bonded to form a catheter.

The method used to make the final prototype biomaterial for testing combined a couple of the methods described above. PDMS monomer, cross-linker and a powdered form of the active materials were combined and thermally cured. To ensure that sufficient amounts of the active materials were exposed at the surface, a percolation threshold was employed. At some ratio, the mass of the powdered metals will be high enough to conduct through the PDMS. If this conduction can be measured at the surface, it is probable that the active materials are available at the surface. This approach can be taken with zinc and silver, however, because silver oxide is not good conductor, the proper ratio was found iteratively through AMR testing, using one or more of known test methods for detecting antimicrobial resistance. A few examples of commonly-used antimicrobial sensitivity testing methods include dilution (broth and agar dilution), disk-diffusion, E-test, mechanism-specific tests such as beta-lactamase detection test and chromogenic cephalosporin test, and genotypic methods such as PCR and DNA hybridization methods.

An important design constraint for urinary catheter coatings is wettability or the ability of a liquid to contact a solid surface due to the intermolecular forces between them. Depending on the ratio of AM:PDMS, changes in surface wettability can also be expected. The wettability of the catheter both influences the biofilm growth on the surface and the comfort of a patient using the catheter. Studies have suggested that interactions between bacterial cell walls and other surfaces are affected by electrostatic interactions and Van der Waals forces. Bacteria tend to have a surface energy smaller than the liquids in which they are suspended and, therefore, attach preferentially to hydrophobic surfaces. Studies regarding the adhesion of different strains of E. coli have also found that they are highly attracted to hydrophobic surfaces, while being slightly repelled by hydrophilic ones. Furthermore, catheters coated with hydrophilic materials also reduce trauma to the urethral surfaces and enable easy and comfortable catheterization for patients, as compared to more hydrophobic conventional catheters. As such, measuring the wettability of the inventive catheter coatings is both important for the prevention of biofilm formation and improvement of patient comfort. One method used to measure wettability is Sessile drop goniometry. The goal is to measure wettability of a surface by measuring the angle that a droplet of liquid forms with a given surface. A low contact angle would indicate a high wettability and a hydrophilic surface, whereas a high contact angle indicates a low wettability and a hydrophobic surface.

The following examples describe the materials and methods used in preparing embodiments of the inventive coating, forming a catheter with the coating and the testing procedures and results for evaluating the coating and devices.

### EXAMPLE 1

### Fabrication of Antimicrobial Film (Thin-Film Composite)

1. PDMS monomer (Dow-Corning, Sylgard™ 184 PDMS), was mixed with 14.3% wt. cross-linker, maximized for tensile properties, and vacuum degassed to remove internal air bubbles;
2. Silver powder (Alpha Aesar, 100 mesh 99.99%) was mixed with silver (I) oxide powder (99.99%) at [2:1] molar ratio in a separate container;
3. The different Active Material/PDMS combinations were weighed and placed in glass beakers separately for each combination and mixed to an even consistency, according to the indicated Active Material:Polydimethylsiloxane (AM:PDMS) mass ratio:
   (1) Zinc:PDMS—4 g:1 g
   (2) Silver:PDMS—3 g:1 g
   (3) Silver (I) oxide:PDMS—1 g:1 g
   (4) Silver/silver (I) oxide:PDMS—3 g:2 g;
   4. Zinc/Silver (I) Oxide:PDMS were prepared by
      (i) mixing together 0.400 g of zinc powder (Sigma Aldrich, 100 mesh 99.99%) with 0.200 g PDMS; and
      (ii) mixing 0.141 g of silver (I) oxide into Zn:PDMS;
   5. A PETG (clear plastic) slide, 250 μm thick with a central well of about 14 mm×60 mm, was adhered to a borosilicate glass slide with drops of 100% ethanol and a strip of Kapton tape;
   6. The AM:PDMS pastes were spread into the well using an additional PETG section;
   7. The mask was removed carefully;
   8. Glass panes were placed in a 125°-150° Celsius oven for 15-20 min to cure;
   9. Samples were diced into rectangles (˜10-12 mm×12-14 mm), or 8 mm discs.
      The sample combinations used in our evaluation are summarized in Table 2 below.

TABLE 2

| | Zn | Ag | Ag₂O |
|---|---|---|---|
| Zn | ✓ | | ✓ |
| Ag | | ✓ | ✓ |
| Ag₂O | ✓ | ✓ | ✓ |

### EXAMPLE 2

### Catheter Preparation—Option 1

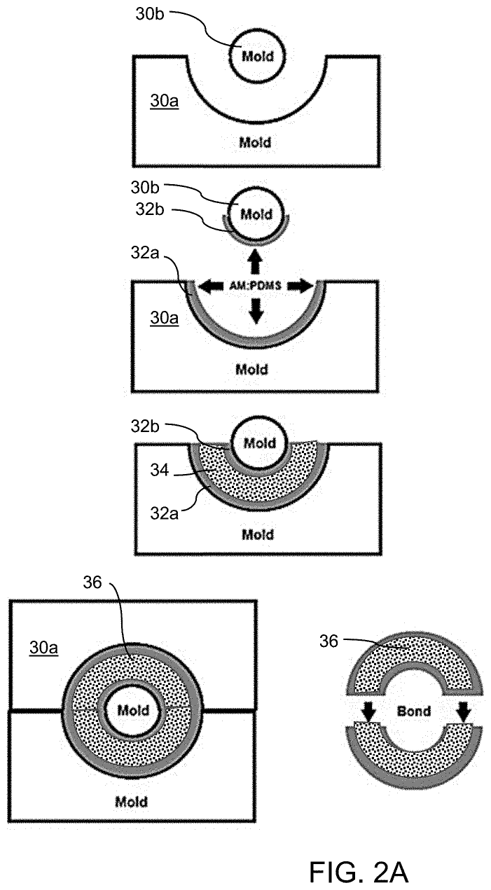

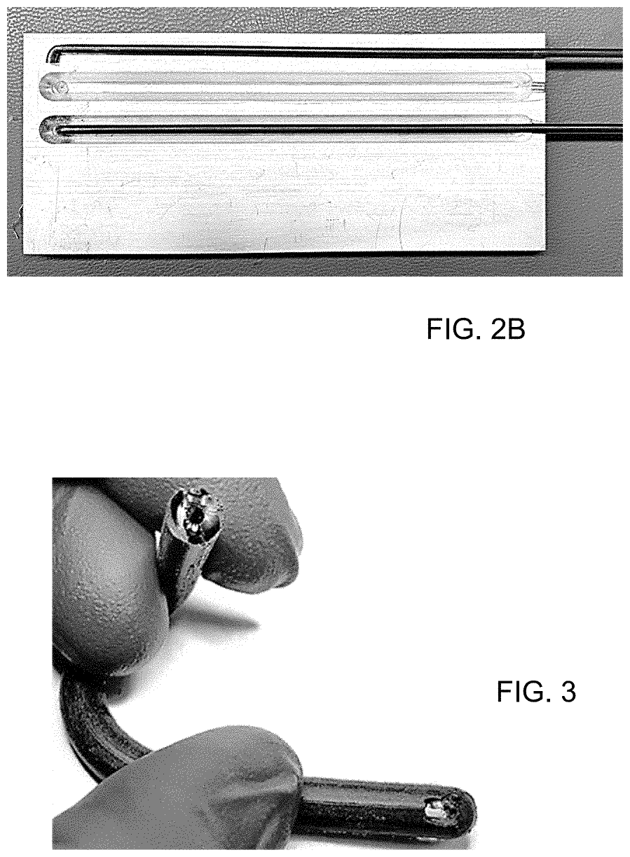

Preparing a complete Foley catheter adds technical challenges. A mold must be prepared to form the main body of the catheter including two lumens/ports for flushing urine and inflating the balloon. One-way valves must be added to the proximal balloon port using a medical grade adhesive. Finally, a balloon must be adhered to the distal end of the catheter. The ratio of active materials to polymer could lead to the material becoming so costly that would not be economical to manufacture, and could degrade its mechanical toughness. To address these issues, a high ratio paste can be made and applied as a thin coating to the surface of a mold over which pure PDMS can be poured/injected. This approach, which is illustrated diagrammatically in FIG. 2A, is able to concentrate the active materials at the surface of the catheter while allowing the bulk of the catheter to be made without the need for the active materials to be incorporated through its full thickness.

The sequence illustrated in FIG. 2A was employed to fabricate the balloon-free, distal end of a catheter. The molds 30a, 30b were machined from aluminum using a 7 mm ball end mill. A 2 mm hole was drilled at the distal tip to support one end of the 2 mm lumen mold, while the other end was supported by the wall of the mold. The overall length of the mold can be on the order of 10-18 cm, as seen in FIG. 2B, which depicts a mold for forming two of the catheter pieces. After the AM:PDMS pastes were prepared as described in Example 1, the molds were heated in an oven at 125° C. for 2 minutes. This allowed the curing process to begin as the pastes 32a, 32b were painted onto the inner surfaces of the molds 30a, 30b. The lumen mold 30b was positioned and pure PDMS 34 was added to fill the remaining space in the mold. The filled molds were cured in an oven at 125° C. for 15 minutes. Each half of the cured catheter 36 was carefully removed from the molds and adhered to one another using a silicone based adhesive (Sil-Poxy™ Smooth-On, Inc.). The distal flush ports were cut/punched using a sharp blade/punch. The resulting device can be seen in the photograph in FIG. 3, shown held by a gloved hand. Following preparation, AM:PDMS pastes can also be coated directly onto a commercial urinary catheter and thermally cured. A variety of methods for applying the coating may be used, including dipping the catheter into the paste, painting the paste onto the catheter, spraying the paste, or other methods as are known in the art.

### EXAMPLE 3

### Catheter Preparation—Option 2

Standard commercially-available silicone catheters without antimicrobial properties were cut to have an outside surface area of 168 mm². The catheters were cleaned using an ultrasonic bath to remove any debris from the surface and secured to a rod set up to be slowly rotated about its axis. A uniform coating of the selected AM:PDMS paste was applied to the outer surface as the rod was rotated. The catheter was heated using a heat gun for about 1 minute while to rod continued to rotate. The rod with the catheter was then placed in an oven at 125° C. for 10 minutes to complete the curing process. The result was a small piece of silicone catheter with a uniform coating thickness of approximately 500 μm.

The foregoing examples describe two possible approaches for catheter preparation. The described options are provided as examples only and are not intended to be limiting. The key is to form a biocompatible flexible polymer coating that has an active material incorporated in a way that at least a portion of the active material is exposed at the polymer surface. Accordingly, variations or combinations of the described examples as well as other methods for forming and applying the inventive antimicrobial coating, will be readily apparent to those of skill in the art.

### EXAMPLE 4

### Testing Materials and Procedures

Synthetic urine was prepared by combining reagents listed in Table 3, and filtering through a 0.2 μm filter.

TABLE 3

| | Quantity | Concentration |
|---|---|---|
| Component | (g) | (mmol/L) |
| Peptone L37 | 1 | |
| Yeast Extract | 0.005 | |
| Lactic Acid | 0.1 | 1.1 |
| Citric Acid | 0.4 | 2.0 |
| Sodium bicarbonate | 2.1 | 25 |
| Urea | 10.0 | 170 |
| Uric acid | 0.07 | 0.4 |
| Creatinine | 0.8 | 7.0 |
| Calcium chloride•2H₂O | 0.37 | 2.5 |
| Sodium chloride | 5.2 | 90 |
| Iron (II) sulfate•7H₂O | 0.0012 | 0.005 |
| Magnesium sulfate•7H₂O | 0.49 | 2.0 |
| Sodium sulphate•10H₂O | 3.2 | 10 |
| Potassium dihydrogen phosphate | 0.95 | 7.0 |
| Di-potassium hydrogen phosphate | 1.2 | 7.0 |
| Ammonium chloride | 1.3 | 25 |

| | |
|---|---|
| Distilled water | To 1 Liter |

In order to explore the quantity of hydrogen peroxide produced by the samples containing the active materials being studied, replicates (n=3) of Zn:PDMS, Ag:PDMS, Ag₂O:PDMS, Ag/Ag₂O:PDMS, Zn/Ag₂O:PDMS, and Zn:PDMS+Ag₂O:PDMS striped pattern specimens were tested over a 13 day period. Samples were sterilized with 100% ethanol and allowed to dry in the bottom of glass containers overnight. While working under a flame, 1.5 mL of synthetic urine was injected into the containers and onto the surface of the sample using a 1 mL syringe. 20 μL of synthetic urine were removed from each sample container and the extracted solution was placed directly on the tile of either Quantofix 25 or Quantofix 100 peroxide strips (Quantofix; Macherey-Nagel, Dueren,

Germany) to reveal a color change used to quantify hydrogen peroxide concentration via comparison to a color scale. Quantofix 25 concentration scale measures: 0, 0.5, 2, 5, 10, and 25 mg/L hydrogen peroxide. Quantofix 100 concentration scale measures: 0, 1, 3, 10, 30, and 100 mg/L hydrogen peroxide. The pH of the urine was measured with litmus paper before being separated into the glass containers, and again for each specimen at the conclusion of the 13 day test.

A standard electrochemical setup with a high impedance electrometer was used to determine the potential difference between silver (Alpha Aesar 99.99%) and zinc (Alpha Aesar 99.9%) foils in air-saturated aqueous solution at room temperature (21° C.) under atmospheric conditions. Synthetic urine was used as the electrolyte for one experiment and DI water for the other. Potential differences were measured using a Solartron 1286 Electrochemical Interface and a two-electrode cell configuration with a 10 mm separation between electrode surfaces. Open circuit potential measurements were obtained at 1 second intervals over a 6 hour period starting immediately after placing the fluid media in the cell.

To determine the wettability of the samples, Sessile drop, contact angle goniometry was performed. The samples were placed under a Keyence VHX 1000 video microscope, positioned at 90 degrees. A 2 μL drop of pure DI water was pipetted onto the separate edges of each sample. The angle is measured using the line and angle measurement tools. Testing was performed on pure PDMS as well as samples made with PDMS mixed with Ag₂O, Ag, Ag/Ag₂O, and Zn/Ag₂O.

### EXAMPLE 5

### Antimicrobial Resistance

48 hour (n=2) and 144 hour (n=5) antimicrobial resistance studies were performed to compare the short-term and long-term effectiveness of selected antimicrobial samples. The samples for the 48 hour short-term study included: PDMS, Ag:PDMS, Zn:PDMS, Ag/Ag₂O:PDMS, Zn/Ag₂O:PDMS, Zn:PDMS+Ag₂O:PDMS striped pattern, and sections of a commercially available “silver-coated” catheter. The samples for the 144 hour long term study included: PDMS, Ag:PDMS, Zn:PDMS, Ag/Ag₂O:PDMS, Zn/Ag₂O:PDMS, Ag₂O:PDMS, and a bioelectric dressing (BED) (Procellera®, Vomaris Innovations, Inc., Tempe, Ariz.), and sections of a commercially available “silver-coated” catheter. A polyester woven cloth served as the control for the BED, and pure PDMS was used as control for the AM:PDMS samples. There was no control for the “silver-coated” catheter. Positive controls for absorbance measurements were test tubes containing only urine and the inoculum. Negative controls for absorbance measurements were test tubes containing only urine without inoculum.

All samples were cut into squares with 168 mm² (12 mm×14 mm) surface area. All samples, apart from the BED and the BED control, were washed with 100% ethanol and placed at the bottom of a test tube to dry overnight. Procellera® and its control were sterilized under UV light for 8 hours on each side, and placed into individual test tubes. The sample-containing test tubes were filled with 3 mL of synthetic urine and inoculated with E. coli (BW 25113) to a known optical density (OD600 =0.01).

The tubes were placed in an incubator at 37° C. without shaking to facilitate growth. The absorbance of each sample was measured using a spectrophotometer (BIORAD SmartSpec™ 3000), at regular intervals. Samples for the 144-hour study were placed into new test tubes containing 3 mL fresh synthetic urine every 48 hours and re-inoculated with bacteria to OD600=0.01 in order to simulate the environmentthat would be present in a urethra.

Biofilm formation was monitored at the end of the short term and long term tests. Sample were placed into a separate sterile glass test tube containing 3 mL distilled water to stunt further growth. The test tubes were then placed in an ultrasonic bath (Branson Ultrasonics CPX Series M 1800) at maximum frequency for three, thirty second intervals, ten seconds apart to shed biofilm from the samples into solution for quantification. A serial dilution was performed until colonies are quantifiable on LB agar plates incubated at 37° C. for 24 hours.

Zone of inhibition studies are a common practice for researchers and pathology labs for testing resistance to antibiotics. The AM:PDMS thin film composites were cut into 8 mm diameter discs using a biopsy tissue punching device. Bacteria were cultured for 24 hours in synthetic urine and coated onto LB 1.5% agar plates. Disks were placed in the center of agar, and plates are incubated overnight at 37° C. Radii of zones of inhibition are measured from center of disk to the edge of bacterial lawn.

Data are plotted using means and standard deviations. The Welch's t-test was used to test the hypothesis that the AM:PDMS coatings are more effective at inhibiting biofilm than just PDMS (placebo) using a confidence interval of 95%.

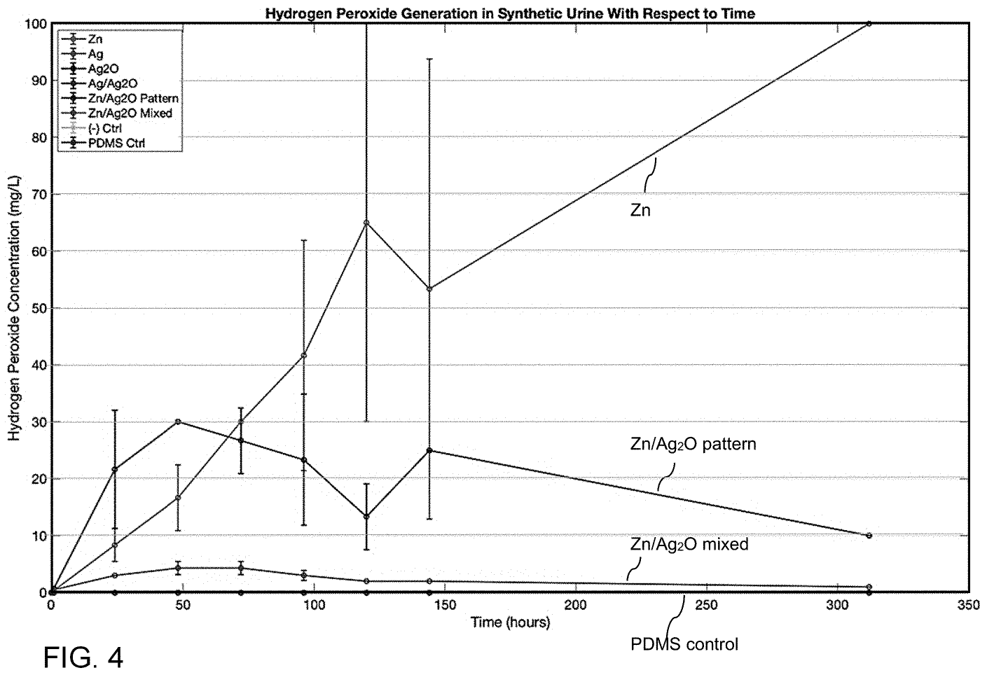

The results shown in FIG. 4 first suggest that only samples containing zinc generate hydrogen peroxide, with samples containing less zinc, i.e., samples combined with silver or silver oxide, producing lower concentrations of the peroxide. None of the other samples generated enough hydrogen peroxide to be detected (<0.5 mg/L). After thirteen days, the pH of the synthetic urine was measured and showed that alkalinity had increased for samples containing zinc and silver oxide, especially for combinations. Table 4 provides the results of pH testing of the different combinations. Three different samples (A, B, C) of each AM were tested.

TABLE 4

| Material (#) | Day 0 | Day 13 |
|---|---|---|
| Control | 7 | 7 |
| PDMS control | 7 | 7 |
| Zn (A) | 7 | 7.5 |
| Zn (B) | 7 | 8 |
| Zn (C) | 7 | 7 |
| Ag (A) | 7 | 7 |
| Ag (B) | 7 | 7 |
| Ag (C) | 7 | 7 |
| Ag₂O (A) | 7 | 7.5 |
| Ag₂O (B) | 7 | 8 |
| Ag₂O (C) | 7 | 8 |
| Ag/Ag₂O (A) | 7 | 7.5 |
| Ag/Ag₂O (B) | 7 | 8 |
| Ag/Ag₂O (C) | 7 | 8.5 |
| Zn/Ag₂O (A) | 7 | 7.5 |
| Zn/Ag₂O (B) | 7 | 8 |
| Zn/Ag₂O (C) | 7 | 7.5 |
| Zn + Ag₂O (A) | 7 | 8 |
| Zn + Ag₂O (B) | 7 | 8 |
| Zn + Ag₂O (C) | 7 | 8 |

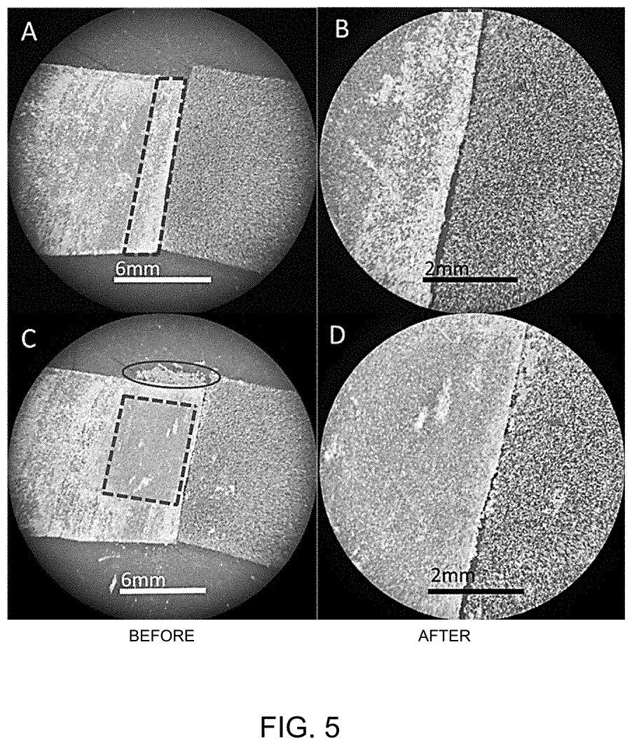

FIG. 5 provides visual evidence of passivation layers building on Zn/Ag₂O:PDMS samples after exposure to synthetic urine for 24 hours. Initially, these samples could be described as a homogenous spectrum of black and gray specks, which can be seen on the right side of each photographic image (panels A-D). After 24 hours in the urine, a white crystal build-up forms on the surfaces of each sample, which can be seen on the left side of each image. Scratching off the build-up reveals a gold hue beneath (region enclosed within the dotted region of panel C, and left side of panel D). It is possible that chloride ions in the urine are reacting with the silver to form silver chloride. It is also possible that zinc is forming zinc hydroxide, i.e., Zn(OH)₂+2e—⇄Zn+2OH—, potential, E⁰: , −1.249V. Both products appear as a white crystalline build-up. However, moistened silver chloride has been known to decompose when exposed to light, turning brown in color. This may explain the discoloration of the sample surface, or perhaps it is result of silver ions reacting with phosphate or sulfate ions found in the urine; silver phosphate appears yellow and silver sulfate darkens upon exposure to air or light.

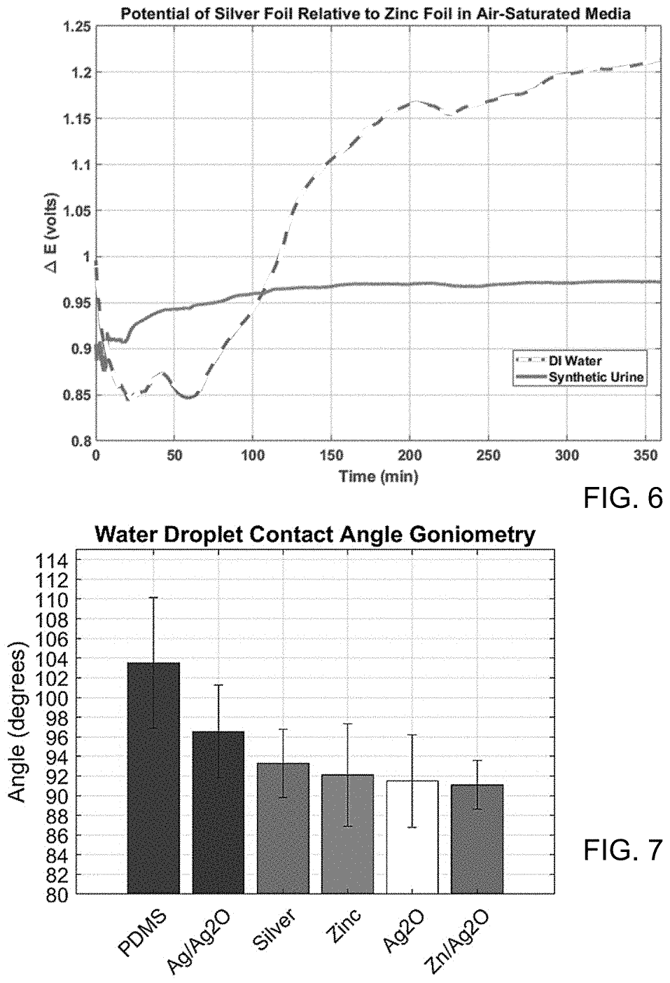

FIG. 6 shows that the potential of silver foil relative to zinc foil changes when the solution is substituted from DI water (dashed line) to the synthetic urine (solid line). The difference between the plateaus of electric potential in the diagram (0.97 Volts and 1.22 Volts) is −0.25 Volts after six hours. This implies establishment of different equilibria at the silver and zinc surfaces when immersed in urine compared to DI water. This information is beneficial as it allows a Pourbaix diagram to be used as a reference, revealing how the active materials may or may not react with dissolved species in urine.

The mean and standard deviation for contact angles of water droplets on each composite and a control are shown as bars in FIG. 7. The graph reveals clear distinctions in surface wettability.

Compared to the pure PDMS surface, all composite samples were more hydrophilic (n=5, p<0.05). Based on the standard deviations, it is difficult to say which composite was more or less wettable, only that they were less than the control. This hydrophilicity is welcomed as it has been shown to resist bacteria adhesion. However, because all samples form water drop contact angles that were greater than 90 degrees, they were all still considered to be slightly hydrophobic or have low wettability.

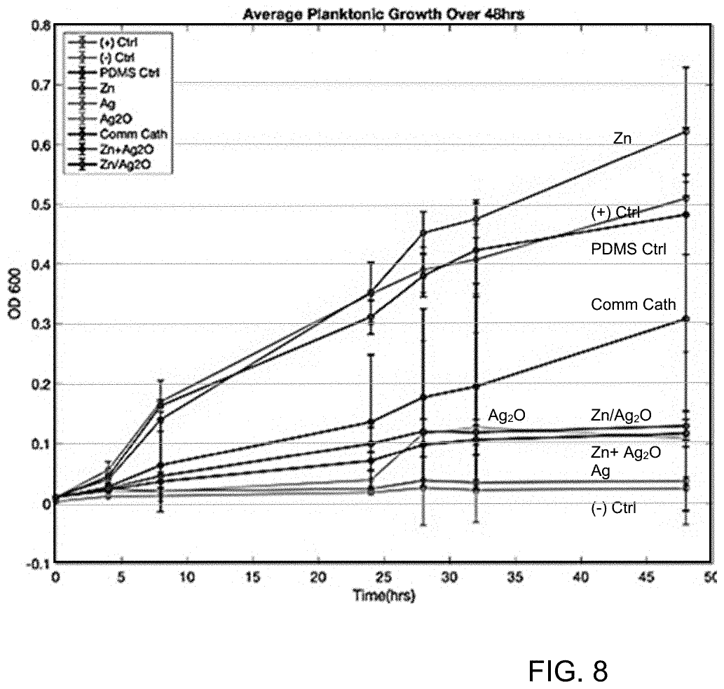

The growth of planktonic bacteria in solution can be tracked using spectrophotometry. The absorbance of light (600 nm) emitted by the machine was measured when passing through a cuvette containing an aliquot of the media containing the inoculum. The optical density (OD600) increases as more light is absorbed by colloid in solution. The sigmoidal curves plotted in FIG. 8 were consistent with that of bacterial growth. Following the first lag cycle, rampant growth can be seen in PDMS, and Zn:PDMS. The commercially-available “silver-coated” catheter (“Comm Cath”) inhibited growth only slightly and to a lesser degree than the samples incorporating the different antimicrobial coatings such as the Zn/Ag₂O:PDMS and the Zn:PDMS+Ag₂O:PDMS striped pattern. The Ag:PDMS exhibited strong antimicrobial effects in solution. This was surprising as elemental silver is less likely to release silver ions into solution.

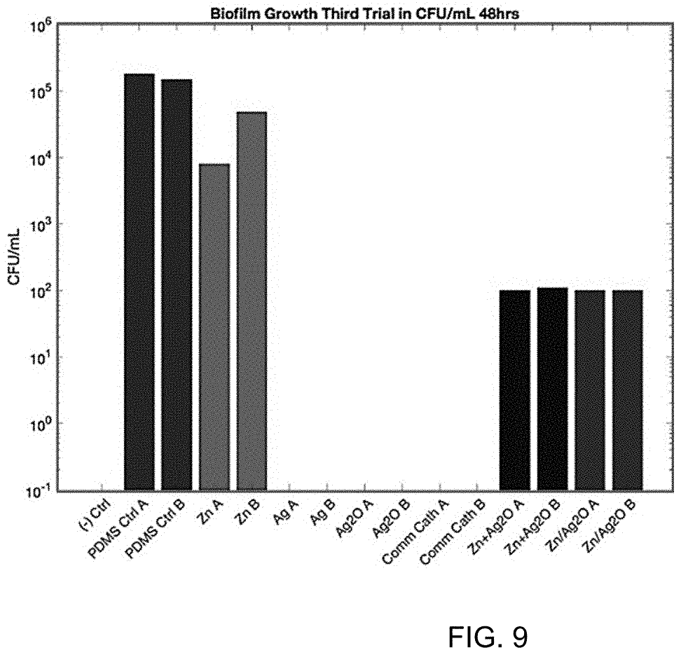

The biofilm shed from samples via ultrasonic bath can be quantified by counting the number of colony forming units (CFUs) that grow from a known volume of solution onto LB agar plates. The number of colonies that form on the plates were counted and used to calculate a concentration using units, CFU/mL. A greater number of colony forming units per milliliter corresponds to a higher number of bacteria growing on the surfaces of the sample. It should be noted that the vertical axis scale for CFU/mL is logarithmic. As shown in FIG. 9, the samples containing zinc had a positive effect on the bacteria, allowing them to grow on the surface. Samples with silver, including the commercial catheter, seemed to perform better for a 48-hour study. When comparing final absorbance magnitudes to the number of CFU/mL, a paradigm is revealed where planktonic bacteria and biofilm are affected independently. For example, the silver catheter inhibits the growth of bacteria moderately within 48 hours but does exceptionally well inhibiting biofilm. The opposite is seen for samples combining zinc and silver oxide. It is difficult to compare samples that equally inhibit bacterial growth, which is why a study lasting six days was conducted.

The long term study was initially designed to last as long as necessary to observe clear distinctions. Following a 48-hour interval, bacteria in the synthetic urine reach a lag cycle. At this point in time, most of the nutrients available to the remaining bacteria in solution are gone and the bacteria begin to be affected by increasing concentrations of their own metabolic byproducts, which can force gene mutations and cell death. For this reason, the synthetic urine was refreshed at 48-hour intervals during the six-day study. This also better mimics in vivo conditions in the bladder as urine is constantly being produced and flushed out of the bladder. Physiologically, the refreshed urine in a urinary tract is expected to still have small concentrations of bacteria present at the time the patient is catheterized. As mentioned previously, the mechanisms by which bacteria are inhibited are complex and can affect growth in solution and on surfaces independently. The study was terminated following 144 hours, when the absorbance of well performing samples approached the magnitude of the controls.

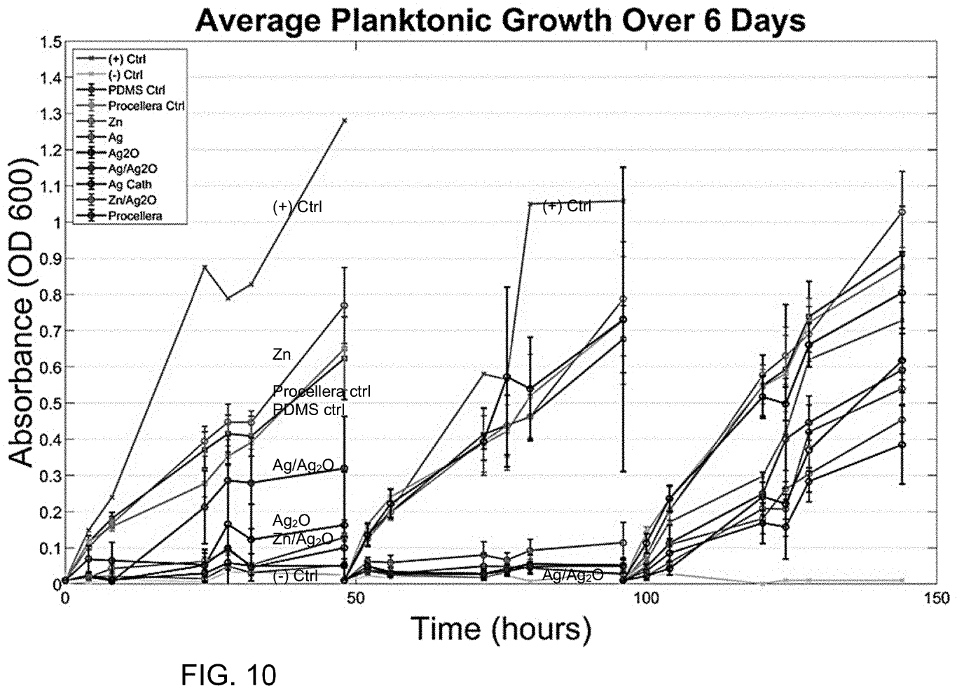

The growth curves following each 48-hour cycle in FIG. 10 exhibit unique differences from sample to sample. During the six days, the positive control shows uncontrolled growth and the negative control shows no growth, as expected. The PDMS sample, the Procellera control, and the Zn:PDMS also show strong growth of E. coli in solution during the six days. During the first 48 hours, Ag/Ag₂O:PDMS is allowing more growth than the remaining samples. After the first 48 hours, the Ag/Ag₂O:PDMS begins to inhibit bacterial growth comparable to the remaining samples, except for the commercially available catheter. It is possible that the commercial silver catheter does in fact have a coating of soluble silver, however, after the urine is refreshed, it appears that there is not enough left to continue inhibiting growth in solution. Not until after the second 48-hour cycle do bacteria become resistant across all samples. It is unclear at what time between 48 hours and 96 hours the samples actually begin to lose their beneficial effect. This is because the E. coli may have all died upon exposure to the films at the start of the second 48-hour cycle, and no further inoculation occurs until hours later.

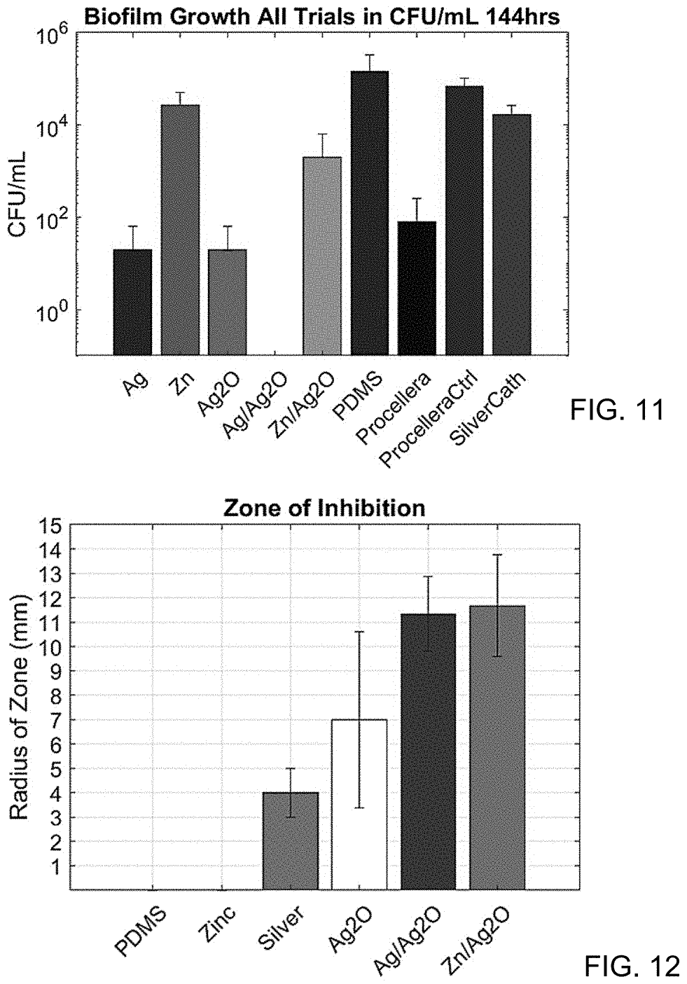

Biofilm was quantified from all samples following repeated exposure to fresh urine and E. coli over six days. The results are shown in FIG. 11. A higher concentration of CFU/mL is directly proportional to a higher concentration of biofilm. Compared to the pure PDMS, Ag:PDMS, Ag₂O:PDMS and Ag/Ag₂O:PDMS were at least five orders of magnitude lower in biofilm (p-value<0.05) . The BED performed slightly worse (p<0.01). The commercial catheter (“SilverCath”) and Zn:PDMS were both ineffective at inhibiting biofilm (p<0.05). Although Zn/Ag₂O:PDMS showed antimicrobial effects in solution, it did not completely prevent biofilm formation. It is possible that the E. coli are using zinc as a nutrient, while silver ions may be the main mechanism behind biofilm inhibition. This is consistent with the fact that all samples containing silver, apart from the commercial silver coated catheter, show lower concentrations of bacteria.

Zone of inhibition results in FIG. 12 provided further insight into the mechanism acting to inhibit bacteria. If bacteria were growing anywhere on the disk (R<4 mm), the sample was unable to inhibit growth. If a zone of inhibition appeared at the edge of the disk (R≈4 mm), then the sample was capable of inhibiting growth about its own surface area. Finally, when a zone of inhibition appeared beyond the edge of the disk (R>4 mm), this suggested that the disk generated a product that could diffuse freely into the agar. What was clear from these results was the importance effect of using a reducible form of silver and/or a strong redox couple. Samples containing only silver (I) oxide varied in how large of a zone was inhibited. Following incorporation of pure silver powder or zinc powder, the diffusive killing effect is much more dramatic.

Samples containing silver (I) oxide were able to inhibit E. coli up to 9 mm away from the edge of the disk. This suggests that these particular films have an improved delivery of a diffusive antimicrobial mechanism.

Comparing the test results, it appears that samples containing reducible forms of silver perform the best in inhibiting planktonic E. coli. Samples made with zinc seemed to lessen the strength on the surface and in solution. This suggests that the e-fields and microcurrents formed between zinc and silver are not significant enough to inhibit biofilm. This also suggests that at the current concentration, zinc could be a nutrient for the E. coli. While this may be the case for E. coli, many other infectious bacteria and fungi are more sensitive to zinc. It is possible that the amount of zinc used to make the Zn/Ag₂O:PDMS samples may have been too high. The goal was to initially load enough zinc to make the coating surface conductive. This proportion of zinc and PDMS was used, including when mixing with silver. To promote a better electrochemical reaction, the amounts of zinc and silver could be varied or patterned in way that improves the electricidal performance. To conclude, these findings may suggest these samples cannot be described as electricidal.

As for the effect of hydrogen peroxide on bacteria, it is apparent that E. coli are indifferent to the concentration of hydrogen peroxide being generated by the samples. The highest concentration of hydrogen peroxide measured was 100 mg/mL or 2.94 millimolar using only zinc. Not only was zinc ineffective at inhibiting the growth of E. coli, so too were high concentrations of hydrogen peroxide. This led to a conclusion that even non-virulent strains of bacteria are capable of protecting themselves from hydrogen peroxide and peroxide anions.

The samples containing silver performed well enough to consider using silver as the main active material for a biocidal catheter. More important than elemental silver is a reducible form of silver, such as silver oxide or silver chloride. These have the ability to participate in reactions with oxygen, hydrogen, hydroxide, and other ions in solution to free silver ions into solution. As is known, silver ions are extremely toxic to bacteria. The elemental silver used to make the composites may have had a thin layer of oxide, similar to the zinc, that initially released silver ions in solution. However, it appears that AMs containing silver oxide are preferred to permit reduction reactions to increase in number or to perpetuate for longer durations of time.

### References—Incorporated Herein by Reference:

[1] C. V Gould, C. A. Umscheid, R. K. Agarwal, G. Kuntz, and D. A. Pegues, “Guideline for Prevention of Catheter-Associated Urinary Tract Infection 2009,” 2017.

[2] C. S. Hollenbeak and A. L. Schilling, “The attributable cost of catheter-associated urinary tract infections in the United States : A systematic review,” AJIC Am. Infect. Control, vol. 46, pp. 751-757, 2018.

[3] D. K. Riley, D. C. Classen, L. E. Stevens, and J. P. Burke, “A Large Randomized Clinical Trial of a Silver-Impregnated Urinary Catheter: Lack of Eikacy and Staphylococcal Superkfection,” Am. J. Med., vol. 98, no. April, pp. 349-356, 1995.

[4] R. M. May et al., “Micro-patterned surfaces reduce bacterial colonization and biofilm formation in vitro: Potential for enhancing endotracheal tube designs,” Clin. Transl. Med., vol. 3, no. 1, p. 8, 2014.

[5] H. Wolfmeier, D. Pletzer, S. C. Mansour, and R. E. W. Hancock, “New Perspectives in Biofilm Eradication,” ACS Infect. Dis., vol. 4, no. 2, pp. 93-106, 2018.

[6] J. Tang et al., “Graphene Oxide—Silver Nanocomposite As a Highly Effective Antibacterial Agent with Species-Specific Mechanisms,” Appl. Mater. Interfaces, vol. 5, pp. 3867-3874, 2013.

[7] A. M. Nandkumar, M. C. Ranjit, S. S. P. Kumar, P. R. Hari, P. Ramesh, and K. Sreenivasan, “Antimicrobial silver oxide incorporated urinary catheters for infection resistance,” Trends Biomater. Artif. Organs, vol. 24, no. 3, pp. 156-164, 2010.

[8] E. L. Lawrence and I. G. Turner, “Materials for urinary catheters: A review of their history and development in the UK,” Med. Eng. Phys., vol. 27, no. 6, pp. 443-453, 2005.

[9] P. Voegele et al., “Antibiofilm activity of electrical current in a catheter model,” Antimicrob. Agents Chemother., vol. 60, no. 3, pp. 1476-1480, 2016.

[10] J. B. Skiba, “Current Producing Surface for Treating Biologic Tissue,” U.S. Pat. No. 7,813,806 B2, 2010.

[11] W. Chiang, C. Schroll, L. R. Hilbert, P. Møller, and T. Tolker-nielsen, “Silver-Palladium Surfaces Inhibit Biofilm Formation,” Appl. Environ. Microbiol., vol. 75, no. 6, pp. 1674-1678, 2009.

[12] J. Banerjee et al., “Silver-Zinc Redox-Coupled electroceutical wound dressing disrupts bacterial biofilm,” PLoS One, vol. 10, no. 3, pp. 1-15, 2015.

[13] J. A. Spadaro, T. J. Berger, S. D. Barranco, S. E. Chapin, and R. O. Becker, “Antibacterial Effects of Silver Electrodes with Weak Direct Current,” Antimicrob. Agents Chemother., vol. 6, no. 5, pp. 637-642, 1974.

[14] J. L. Del Pozo, M. S. Rouse, J. N. Mandrekar, J. M. Steckelberg, and R. Patel, “The electricidal effect: Reduction of Staphylococcus and Pseudomonas biofilms by prolonged exposure to low-intensity electrical current,” Antimicrob. Agents Chemother., vol. 53, no. 1, pp. 41-45, 2009.

[15] P. Singha, J. Locklin, and H. Handa, “A review of the recent advances in antimicrobial coatings for urinary catheters,” Acta Biomater., vol. 50, pp. 20-40, 2017.

[16] S. Sarangapani, “Encrustation and Bacterial Resistant Coatings for Medical Applications,” U.S. Pat. No. 5,877,243, 1999.

[17] M. Alagiri, “Personal Interview.” University of California San Diego, 2018.

[18] H. H. Tuson and D. B. Weibel, “Bacteria-surface interactions,” Soft Matter, vol. 9, no. 17, pp. 4368-4380, 2013.

[19] D. Davies, “Understanding Biofilm Resistance to Antimicrobial Agents,” Nat. Rev. Drug Discov., vol. 2, no. February, pp. 114-122, 2003.

[20] J. Banerjee et al., “Improvement of human keratinocyte migration by a redox active bioelectric dressing,” PLoS One, vol. 9, no. 3, 2014.

[21] O. Wahlsten, J. Skiba, I. Makin, and P. Apell, “Electrical field landscape of two electroceuticals,” J. Electr. Bioimpedance, vol. 7, no. 1, p. 13, 2016.

[22] R. V. Andino, C. J. Brooks, M. J. Keating, C. F. Morgan, and D. Van Royen, “Apparatus and Methods for Facilitating Wound Healing and Treating Skin,” US 2008/0027509 A1, 2008.

[23] C. Morris, “Bio-electrical stimulation therapy using POSiFECT® RD,” Wounds UK, vol. 2, no. 4, pp. 112-115, 2006.

[24] C. D. Bernanier, E. B. Feldman, W. P. Flatt, and S. T. St. Jeor, Eds., Handbook of Nutrition and Food. Boca Raton: CRC Press, 2002.

[25] Z. Li, X. Gu, S. Lou, and Y. Zheng, “The development of binary Mg-Ca alloys for use as biodegradable materials within bone,” Biomaterials, vol. 29, no. 10, pp. 1329-1344, 2008.

[26] X. Gu, Y. Zheng, Y. Cheng, S. Zhong, and T. Xi, “In vitro corrosion and biocompatibility of binary magnesium alloys,” Biomaterials, vol. 30, no. 4, pp. 484-498, 2009.

[27] L. M. Plum, L. Rink, and H. Haase, “The Essential Toxin: Impact of Zinc on Human Health,”Int. J. Environ. Res. Public Health, vol. 7, pp. 1342-1365, 2010.

[28] J. Sawai et al., “Hydrogen Peroxide as a Antibacterial Factor in Zinc Oxide Powder Slurry,” J. Ferment. Bioeng., vol. 86, no. 5, pp. 521-522, 1998.

[29] C. E. Bayliss and W. M. Waites, “The Effect of Hydrogen Peroxide and Ultraviolet Irradiation on Non-sporing Bacteria,” J. Appl. Bacteriol., vol. 48, pp. 417-422, 1980.

[30] D. Goodsell, “Molecule of the Month: Catalase,” PDB-101, 2004. [Online]. Available: http://pdb101.rcsb.org/motm/57. [Accessed: 17 Feb. 2019].

[31] J. R. J. Rumble, Ed., CRC Handbook of Chemistry and Physics, 100th ed. Boca Raton, Fla.: CRC Press, Taylor & Francis Group, 2019.

[32] A. J. Bard and L. R. Faulkner, Electrochemical Methods: Fundamentals and Applications, 2nd ed. New York, New York, USA: John Wiley & Sons, 2001.

[33] M. Pourbaix, Atlas of Electrochemical Equilibria in Aqueous Solutions, 2nd ed. Houston, Tex., USA: National Association of Corrosion Engineers, 1974.

[34] L. W. Mckeen, “Plastics Used in Medical Devices,” in Handbook of Polymer Applications in Medicine and Medical Devices, Elsevier Inc., 2014, pp. 21-54.

[35] A. Mata, A. J. Fleischman, and S. Roy, “Characterization of polydimethylsiloxane (PDMS) properties for biomedical micro/nanosystems.,” Biomed. Microdevices, vol. 7, no. 4, pp. 281-293, 2005.

[36] S. Berchmans, A. J. Bandodkar, W. Jia, J. Ram, Y. S. Meng, and J. Wang, “An epidermal alkaline rechargeable Ag—Zn printable tattoo battery for wearable electronics †,” J. Mater. Chem. A, vol. 2, pp. 15788-15795, 2014.

[37] R. Vilkhu, W. J. C. Thio, P. Das Ghatak, C. K. Sen, A. C. Co, and A. Kiourti, “Power Generation for Wearable Electronics: Designing Electrochemical Storage on Fabrics,” IEEE Access, vol. 6, pp. 28945-28950, 2018.

[38] K. Choi, D. B. Ahn, and S. Lee, “Current Status and Challenges in Printed Batteries: Toward Form Factor-Free, Monolithic Integrated Power Sources,” ACS Energy Lett., vol. 3, pp. 220-236, 2018.

[39] G. A. Ghiurcan, C. Liu, A. Webber, and F. H. Feddrix, “Development and Characterization of a Thick-Film Printed Zinc-Alkaline Battery,” J. Electrochem. Soc., vol. 150, no. 7, pp. 922-927, 2003.

[40] R. Kumar, J. Shin, L. Yin, J. You, Y. S. Meng, and J. Wang, “All-Printed, Stretchable Zn-Ag 2 O Rechargeable Battery via Hyperelastic Binder for Self-Powering Wearable Electronics,” Adv. Energy Mater., vol. 7, no. 1602096, p. 8pp, 2017.

[41] K. T. Braam, S. K. Volkman, and V. Subramanian, “Characterization and optimization of a printed, primary silver—zinc battery,” J. Power Sources, vol. 199, pp. 367-372, 2012.

[42] L. Picard, P. Phalip, E. Fleury, and F. Ganachaud, “Chemical adhesion of silicone elastomers on primed metal surfaces: A comprehensive survey of open and patent literatures,” Prog. Org. Coatings, vol. 80, pp. 120-141, 2015.

[43] A. Goyal et al., “In situ Synthesis of Metal Nanoparticle Embedded Free Standing Multifunctional PDMS Films,” Macromol. Rapid Commun., vol. 30, pp. 1116-1122, 2009.

[44] D. Koh, A. Wang, P. Shneider, B. Bosinski, and K. W. Oh, “Introduction of a Chemical-Free Metal PDMS Thermal Bonding for Fabrication of Flexible Electrode by Metal Transfer onto PDMS,” Micromachines, vol. 8, no. 9, p. 280, 2017.

[45] M. E. Spahr, D. Goers, A. Leone, S. Stallone, and E. Grivei, “Development of carbon conductive additives for advanced lithium ion batteries,” J. Power Sources, vol. 196, pp. 3404-3413, 2011.

[46] S. Vadukumpully, J. Paul, N. Mahanta, and S. Valiyaveettil, “Flexible conductive graphene/poly(vinyl chloride) composite thin films with high mechanical strength and thermal stability,” Carbon N. Y., vol. 49, pp. 198-205, 2011.

[47] J. Clerc, G. Giraud, S. Alexander, and E. Guyon, “Conductivity of a Mixture of Conducting Insulating Grains: Dimensionality Effects,” Am. Phys. Soc., vol. 22, no. 5, pp. 2489-2494, 1980.

[48] L. D. Renner and D. B. Weibel, “NIH Public Access,” MRS Bull., vol. 36, no. 5, pp. 347-355, 2011.

[49] Y. Ong, A. Razatos, G. Georgiou, and M. M. Sharma, “Adhesion Forces between E. coli Bacteria and Biomaterial Surfaces,” Langmuir, vol. 15, no. 8, pp. 2719-2725, 1999.

[50] S. Sarica, Y. Akkoc, H. Karapolat, and H. Aktug, “Comparison of the use of conventional, hydrophilic and gel-lubricated catheters with regard to urethral micro trauma, urinary system infection, and patient satisfaction in patients with spinal cord injury: a randomized controlled study,” Eur. J. Phys. Rehabil. Med., vol. 46, no. 4, pp. 473-480, 2010.

[51] T. Brooks and C. W. Keevil, “A simple artificial urine for the growth of urinary pathogens,”Lett. Appl. Microbiol., vol. 24, pp. 203-206, 1997.

[52] I. E. C. Mott, D. J. Stickler, W. T. Coakley, and T. R. Bott, “The removal of bacterial biofilm from water-filled tubes using axially propagated ultrasound,” J. Appl. Microbiol., vol. 84, pp. 509-514, 1998.

[53] G. E. F. Lundell and J. I. Hoffman, “The Effect of Light on Silver Chloride in Chemica,”Bur. Stand. J. Res., vol. 4, no. RP 134, pp. 0-5, 1929.

1. An antimicrobial coating comprising:
a biocompatible flexible polymer having incorporated therein an active material comprising a reducible form of silver, wherein at least a portion of the active material is exposed at a surface of the polymer.

2. The antimicrobial coating of claim 1, wherein the flexible polymer is polydimethylsiloxane (PDMS).

3. The antimicrobial coating of claim 1, wherein the active material comprises silver oxide or silver chloride.

4. The antimicrobial coating of claim 3, wherein the active material further comprises one or more of zinc and silver.

5. The antimicrobial coating of claim 1, wherein the active material is incorporated into the polymer by forming a paste from a monomer, a cross-linker and a powder of the active material and thermally curing the paste.

6. The antimicrobial coating of claim 5, wherein the paste is filled into a mold configured to form a lumen.

7. The antimicrobial coating of claim 5, wherein the paste is applied to an outer surface of a catheter prior to thermally curing.

8. A method for inhibiting growth and proliferation of biofilm on a medical device, comprising applying the antimicrobial coating of any one of claims 1 to 7 to surfaces of the medical device, wherein the coating is configured to induce an electrochemical/galvanic reaction on the device surfaces when exposed to an electrolyte comprising bodily fluids.

9. A catheter lumen comprising:
a biocompatible flexible polymer film having incorporated therein an active material comprising a reducible form of silver, wherein at least a portion of the active material is exposed at surface of the polymer, wherein the active material is configured to induce an electrochemical/galvanic reaction when exposed to an electrolyte comprising bodily fluids.

10. The catheter lumen of claim 9, wherein the flexible polymer is polydimethylsiloxane (PDMS).

11. The catheter lumen of claim 9, wherein the active material comprises silver oxide or silver chloride.

12. The catheter lumen of claim 11, wherein the active material further comprises one or more of zinc and silver.

13. The catheter lumen of claim 9, wherein the active material is incorporated into the polymer by forming a paste from a monomer, a cross-linker and a powder of the active material and thermally curing the paste.

14. The catheter lumen of claim 13, wherein the paste is filled into a mold configured to form a lumen.

15. The catheter lumen of claim 13, wherein the paste is applied to an outer surface of a catheter prior to thermally curing.
